(19) United States
(12) Patent Application Publication
(10) Pub. No.: US 2006/0105979 A1
(43) Pub. Date: May 18, 2006

(54) Synergistic inhibition of VEGF and modulation of the immune response

(75) Inventors: Sudhir Agrawal, Shrewsbury, MA (US); Ekambar R. Kandimalla, Southboro, MA (US)

Correspondence Address: Wayne A. Keown, Ph. D.;Keown & Associates, Suite 1200, 500 West Cummings Park, Woburn, MA, 01801, US

(73) Assignee: Hybridon, Inc.

(21) Appl. No.: 11/269,862

(22) Filed: Nov. 8, 2005

Related U.S. Application Data
(60) Provisional application No. 60/625,844, filed on Nov. 8, 2004.

(51) Int. Cl. A61K 48/00

(52) U.S. Cl. 514/44

(57) ABSTRACT

The invention provides methods and compositions for treating asthma and allergy by inhibiting VEGF expression and modulating the immune system from a Th2 response to a Th1 response.

This application claims the benefit of U.S. Provisional Application No. 60/625,844, filed Nov. 8, 2004.

### BACKGROUND OF THE INVENTION

1. Field of the Invention

The invention relates to the inhibition of vascular endothelial growth factor and modulation of the immune response. More particularly, the invention relates to the use of such inhibition and modulation for the treatment of disease.

2. Summary of the Related Art

Angiogenesis, the growth of new capillaries from pre-existing vessels, contributes to the development and progression of a variety of physio-pathological conditions. There is growing evidence that anti-angiogenic drugs will improve future therapies of diseases like cancer, rheumatoid arthritis and ocular neovascularisation. Conversely, therapeutic angiogenesis is an important homeostatic response contributing to limit the damage to ischemic tissues. Molecular processes involved in angiogenesis include stimulation of endothelial growth by cytokine production (e.g. vascular endothelial growth factor, VEGF), degradation of extracellular matrix proteins by matrix metalloproteinases (MMPs), and migration of endothelial cells mediated by integrins (cell membrane adhesion molecules). Vascular endothelial growth factor (VEGF), which was originally discovered as vascular permeability factor, is critical to human cancer angiogenesis through its potent functions as a stimulator of endothelial cell survival, mitogenesis, migration, differentiation and self-assembly, as well as vascular permeability, immunosuppression and mobilization of endothelial progenitor cells from the bone marrow into the peripheral circulation.

Hoshino, M., et al., J. Allergy Clin. Immunol. 107, 1034-1038 (2001); J. Allergy Clin. Immunol. 107, 295-301 (2001) teaches that overexpression of VEGF has been detected in tissues and biological samples from people suffering from asthma and the levels of VEGF have been directly correlated with asthma. Lee, Y.C. et al., J. Allergy Clin. Immunol. 107, 1106-1108 (2001) teaches that VEGF directly contributes to the pathogenesis of asthma phenotype. Lee et al., Nature Medicine 10: 1095-1103 (2004) teaches that transgenic mice overexpressing VEGF demonstrate that VEGF potently stimulates angiogenesis, edema, inflammation, vascular remodeling, parenchymal remodeling and augments antigen sensitization and Th2 inflammation. Lee et al., Nature Medicine 10: 1095-1103 (2004) teaches that VEGF production is a critical event in Th2 inflammation, through both IL-13-dependent and-independent pathways, and Th2 cytokine elaboration in antigen sensitized mouse lungs.

Antisense and siRNA methods have been shown to be attractive approaches to down regulating unwanted gene expression in vitro and in vivo. Robinson GS. Et al., Proc. Natl. Acad. Sci. USA. 93, 4851-4856 (1996); Masood R. et al., Proc. Natl. Acad. Sci. USA. 94, 979-984 (1997); Takei, Y. et al., Cancer Res. 64, 3365-3370 (2004) teach that VEGF is an attractive target for antisense and siRNA drug development for angiogenic disorders such as cancer, age-related macular degeneration and diabetic-retinopathy. Robinson GS. Et al., Proc. Natl. Acad. Sci. USA. 93, 4851-4856 (1996); Masood R. et al., Proc. Natl. Acad. Sci. USA. 94, 979-984 (1997) teach the use of VEGF antisense agents as anticancer drugs and as therapies for macular degeneration. These VEGF antisense agents may also be useful in treating VEGF-induced Th2 inflammation in antigen-sensitized lungs.

Many kinds of immune cells and mediators contribute to the exacerbations and progress of allergy and asthma. Therefore, there are numerous potential modalities to treat the disease. Umetsu DT, et al., Nature Immunol. 3, 715-720 (2002)teach that the dynamic of the Th1/Th2 phenotype as it relates to allergy and asthma is important, and that modulation of this balance with the goal of suppressing Th2 responses may be useful in treating these diseases. However, Bharadwaj, A. et al., Int. Immunopharmacol. 4, 495-511 (2004) teach that stimulation of antigen-specific Th1 responses is also necessary for proper suppression of Th2 responses, as the T-helper subsets are polarizing and mutually antagonistic in nature. Agrawal S. et al., Ann. N. Y. Acad. Sci. 1002, 30-42 (2003) teaches that synthetic oligodeoxynucleotides containing unmethylated CpG, YpG, CpR, R'pG, YpR dinucleotides (immunomodulatory oligonucleotides, IMOs) act as TLR9 agonists and can potently stimulate innate immune responses and thereby acquired immunity. Zhu F. G. et al., Int. Immunopharmacol. 4, 851-862 (2004) and Agrawal D. K., et al., Int. Immunopharmacol. 4, 127-138 (2004) teach that decreases in IL-4, IL-5, IL-13, IgE and eosinophilia and increases in IL-12, IFN-_{γ}, IgG2a have been observed in mouse models using IMOs. Zhu F. G. et al., Int. Immunopharmacol. 4, 851-862 (2004) and Agrawal D. K., et al., Int. Immunopharmacol. 4, 127-138 (2004) teaches that IMOs prevent and reverse antigen-induced Th2 immune responses in mouse models.

There remains a need for new and more effective methods for treating allergy and asthma.

### BRIEF SUMMARY OF THE INVENTION

The invention provides new and more effective methods and compositions for treating allergy and asthma. The present inventors have surprisingly discovered that it is beneficial to use the combination of VEGF antisense/siRNA to directly suppress VEGF promoted Th2 immune responses in combination with IMOs to enhance Th1 immune responses for the treatment of asthma and allergies.

Thus, in a first aspect, the invention provides a method for treating asthma and/or allergies. The method according to the invention comprises administering to a mammal having allergies and/or asthma a therapeutically effective amount of a VEGF expression-inhibiting antisense oligonucleotide and/or siRNA in combination with a therapeutically effective amount of an IMO.

In a second aspect, the invention provides a compostion of matter comprising a therapeutically effective amount of a VEGF expression-inhibiting antisense oligonucleotide and/or siRNA and a therapeutically effective amount of an IMO.

In a third aspect, the invention provides a pharmaceutical formulation comprising a therapeutically effective amount of a VEGF expression-inhibiting anti sense oligonucleotide and/or siRNA, a therapeutically effective amount of an IMO and a pharmaceutically acceptable carrier or diluent.

### BRIEF DESCRIPTION OF THE DRAWINGS

FIG. 1 shows an embodiment of a treatment protocol using ovalbumin, antisense complementary to VEGF RNA and an immunomodulatory oligonucleotide.

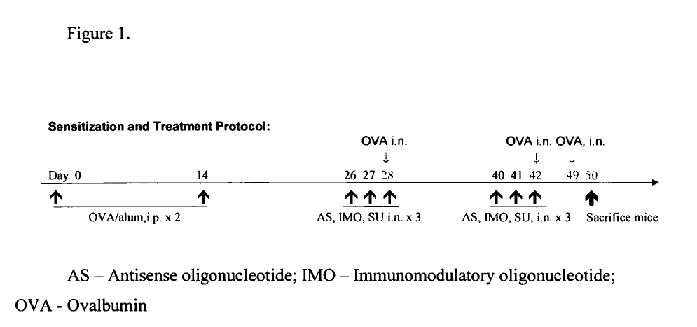

FIG. 2 shows an embodiment of a prophylactic protocol using ovalbumin, antisense complementary to VEGF RNA and an immunomodulatory oligonucleotide.

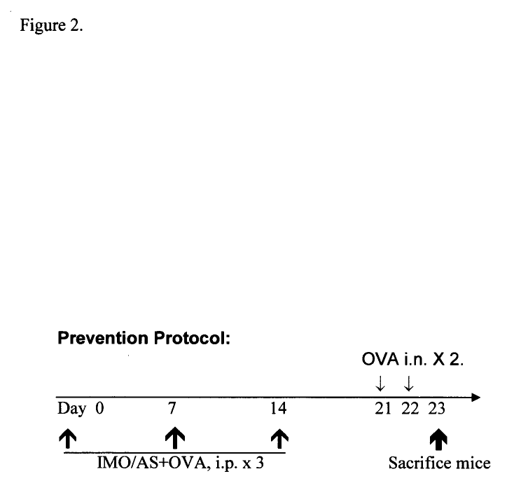

### DETAILED DESCRIPTION OF THE PREFERRED EMBODIMENTS

The invention relates to the inhibition of vascular endothelial growth factor. More particularly, the invention relates to the use of such inhibition for the treatment of disease. The invention provides new and more effective methods and compositions for treating allergy and asthma.

In a first aspect, the invention provides a method for treating asthma and/or allergies. The method according to the invention comprises administering to a mammal having allergies and/or asthma a therapeutically effective amount of a VEGF expression-inhibiting antisense oligonucleotide and/or siRNA in combination with a therapeutically effective amount of an IMO.

For purposes of the invention, the term “oligonucleotide” refers to a polynucleoside formed from a plurality of linked nucleoside units. Such oligonucleotides can be obtained from existing nucleic acid sources, including genomic or cDNA, but are preferably produced by synthetic methods. In preferred embodiments each nucleoside unit includes a heterocyclic base and a pentofuranosyl, trehalose, arabinose, 2′-deoxy-2′-subsituted arabinose, 2′-O-substituted arabinose or hexose sugar group. The nucleoside residues can be coupled to each other by any of the numerous known intemucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific intemucleoside linkage (e.g., (R_{P})— or (S_{P})-phosphorothioate, alkylphosphonate, or phosphotriester linkages). As used herein, the terms “oligonucleotide” and “dinucleotide” are expressly intended to include polynucleosides and dinucleosides having any such intemucleoside linkage, whether or not the linkage comprises a phosphate group. In certain preferred embodiments, these internucleoside linkages may be phosphodiester, phosphorothioate, or phosphorodithioate linkages, or combinations thereof.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), locked nucleic acids (LNA), morpholino-backbone oligonucleotides , and oligonucleotides having backbone sections with alkyl linkers or amino linkers.

The oligonucleotides of the invention can include naturally occurring nucleosides, modified nucleosides, or mixtures thereof. As used herein, the term “modified nucleoside” is a nucleoside that includes a modified heterocyclic base, a modified sugar moiety, or a combination thereof. In some embodiments, the modified nucleoside is a non-natural pyrimidine or purine nucleoside, as herein described. In some embodiments, the modified nucleoside is a 2′-substituted ribonucleoside an arabinonucleoside or a 2′-deoxy-2′-flouroarabinoside.

For purposes of the invention, an IMO is an oligonucleotide or oligonucleotide analog having an immunomodulatory dinucleotide. In preferred embodiments, the immunomodulatory dinucleotide is selected from the group consisting of CpG, YpG, CpR, and YpR, R₁pG and R₁pR wherein C is cytidine or 2′-deoxycytidine, Y is 5-hydroxy-C, arabinocytidine, 2′-deoxy-2′-substituted arabinocytidine, 2′-O-substituted arabinocytidine, 2′-deoxy-5-hydroxycytidine, 2′-deoxy-N4-alkyl-cytidine, N4-alkyl-cytidine, 2′-deoxy-4-thiouridine or other non-natural pyrimidine nucleoside, G is guanosine or 2′-deoxyguanosine, R is 2′ deoxy-7-deazaguanosine, 2′-deoxy-6-thioguanosine, arabinoguanosine, 2′-deoxy-2′substituted-arabinoguanosine, 2′-O-subsituted-arabinoguanosine, 2′-deoxyinosine, or other non-natural purine nucleoside, R₁ is (1-(2-deoxy-D-ribofuranosyl)-2-oxo-7-deaza-8-methyl-purine, and p is an intemucleoside linkage selected from the group consisting of phosphodiester, phosphorothioate, and phosphorodithioate. In certain preferred embodiments, the immunostimulatory dinucleotide is not CpG.

The immunomodulatory oligonucleotides may include immunostimulatory moieties on one or both sides of the immunostimulatory dinucleotide. Thus, in some embodiments, the immunomodulatory oligonucleotide comprises an immunostimulatory domain of the structure:
5′-Nn-N1-Y-Z-N1-Nn-3′

wherein:

Y is cytidine, 2′deoxythymidine, 2′ deoxycytidine arabinocytidine, 2′-deoxy-2′-substitutedarabinocytidine 2′-deoxythymidine, 2′-O-substitutedarabinocytidine, 2′-deoxy-5-hydroxycytidine, 2′-deoxy-N4-alkyl-cytidine, 2′-deoxy-4-thiouridine or other non-natural pyrimidine nucleoside;

Z is guanosine or 2′-deoxyguanosine, 2′ deoxy-7-deazaguanosine, 2′-deoxy-6-thioguanosine, arabinoguanosine, 2′-deoxy-2′substituted-arabinoguanosine, 2′-O-substituted-arabinoguanosine, 2′deoxyinosine, or other non-natural purine nucleoside;

N1, at each occurrence, is preferably a naturally occurring or a synthetic nucleoside or an immunostimulatory moiety selected from the group consisting of abasic nucleosides, arabinonucleosides, 2′-deoxyuridine, α-deoxyribonucleosides, β-L-deoxyribonucleosides, and nucleosides linked by a phosphodiester or modified intemucleoside linkage to the adjacent nucleoside on the 3′ side, the modified intemucleotide linkage being selected from, without limitation, a linker having a length of from about 2 angstroms to about 200 angstroms, C2-C18 alkyl linker, poly(ethylene glycol) linker, 2-aminobutyl-1,3-propanediol linker, glyceryl linker, 2′-5′ intemucleoside linkage, and phosphorothioate, phosphorodithioate, or methylphosphonate intemucleoside linkage;

Nn, at each occurrence, is preferably a naturally occurring nucleoside or an immunostimulatory moiety selected from the group consisting of abasic nucleosides, arabinonucleosides, 2′-deoxyuridine, a-deoxyribonucleosides, 2′-O-substituted ribonucleosides, and nucleosides linked by a modified intemucleoside linkage to the adjacent nucleoside on the 3′ side, the modified intemucleotide linkage preferably being selected from the group consisting of amino linker, 2′-5′ intemucleoside linkage, and methylphosphonate intemucleoside linkage;

provided that at least one N1 or Nn is an immunostimulatory moiety;

wherein n is a number from 0 to 30; and

wherein the 3′ end, an intemucleoside linker, or a derivatized nucleobase or sugar is linked directly or via a non-nucleotidic linker to another oligonucleotide, which may or may not be immunostimulatory.

In some preferred embodiments, YZ is arabinocytidine or 2′-deoxy-2′-substituted arabinocytidine and arabinoguanosine or 2′deoxy-2′-substituted arabinoguanosine. Preferred immunostimulatory moieties include modifications in the phosphate backbones, including, without limitation, methylphosphonates, methylphosphonothioates, phosphotriesters, phosphothiotriesters, phosphorothioates, phosphorodithioates, triester prodrugs, sulfones, sulfonamides, sulfamates, formacetal, N-methylhydroxylamine, carbonate, carbamate, morpholino, boranophosphonate, phosphoramidates, especially primary amino-phosphoramidates, N3 phosphoramidates and N5 phosphoramidates, and stereospecific linkages (e.g., (R_{P})— or (S_{P})-phosphorothioate, alkylphosphonate, or phosphotriester linkages).

Preferred immunomodulatory moieties according to the invention further include nucleosides having sugar modifications, including, without limitation, 2′-substituted pentose sugars including, without limitation, 2′-O-methylribose, 2′-O-methoxyethyl-ribose, 2′-O-propargylribose, and 2′-deoxy-2′-fluororibose; 3′-substituted pentose sugars, including, without limitation, 3′-O-methylribose; 1′,2′-dideoxyribose; arabinose; substituted arabinose sugars, including, without limitation, 1′-methylarabinose, 3′-hydroxymethylarabinose, 4′-hydroxymethylarabinose, and 2′-substituted arabinose sugars; hexose sugars, including, without limitation, 1,5-anhydrohexitol; and alpha-anomers. In embodiments in which the modified sugar is a 3′-deoxyribonucleoside or a 3′-O-substituted ribonucleoside, the immunostimulatory moiety is attached to the adjacent nucleoside by way of a 2′-5′ intemucleoside linkage.

Preferred immunomodulatory moieties according to the invention further include oligonucleotides having other carbohydrate backbone modifications and replacements, including peptide nucleic acids (PNA), peptide nucleic acids with phosphate groups (PHONA), locked nucleic acids (LNA), morpholino backbone oligonucleotides, and oligonucleotides having backbone linker sections having a length of from about 2 angstroms to about 200 angstroms, including without limitation, alkyl linkers or amino linkers. The alkyl linker may be branched or unbranched, substituted or unsubstituted, and chirally pure or a racemic mixture. Most preferably, such alkyl linkers have from about 2 to about 18 carbon atoms. In some preferred embodiments such alkyl linkers have from about 3 to about 9 carbon atoms. Some alkyl linkers include one or more functional groups selected from the group consisting of hydroxy, amino, thiol, thioether, ether, amide, thioamide, ester, urea, and thioether. Some such functionalized alkyl linkers are poly(ethylene glycol) linkers of formula —O—(CH₂—CH₂—O—)ₙ (n=1-9). Some other functionalized alkyl linkers are peptides or amino acids.

Preferred immunomodulatory moieties according to the invention further include DNA isoforms, including, without limitation, β-L-deoxyribonucleosides and α-deoxyribonucleosides. Preferred immunomodulatory moieties according to the invention incorporate 3′ modifications, and further include nucleosides having unnatural intemucleoside linkage positions, including, without limitation, 2′-5′, 2′-2′, 3′-3′ and 5′-5′ linkages.

Preferred immunomodulatory moieties according to the invention further include nucleosides having modified heterocyclic bases, including, without limitation, 5-hydroxycytosine, 5-hydroxymethylcytosine, N4-alkylcytosine, preferably N4-ethylcytosine, 4-thiouracil, 6-thioguanine, 7-deazaguanine, inosine, nitropyrrole, C5-propynylpyrimidine, and diaminopurines, including, without limitation, 2,6-diaminopurine.

IMOs useful in the invention also include immunomers. “Immunomers” comprise at least two oligonucleotides linked at their 3′ ends or intemucleoside linkage or a functionalized nucleobase or sugar via a non-nucleotidic linker, wherein at least one oligonucleotide is an IMO. For purposes of the invention, a “non-nucleotidic linker” is any moiety that can be linked to the oligonucleotides by way of covalent or non-covalent linkages. Preferably such linker is from about 2 angstroms to about 200 angstroms in length. Several examples of preferred linkers are set forth below. Non-covalent linkages include, but are not limited to, electrostatic interaction, hydrophobic interactions, π-stacking interactions, and hydrogen bonding. The term “non-nucleotidic linker” is not meant to refer to an internucleoside linkage, as described above, e.g., a phosphodiester, phosphorothioate, or phosphorodithioate functional group, that directly connects the 3′-hydroxyl groups of two nucleosides. For purposes of this invention, such a direct 3′-3′ linkage is considered to be a “nucleotidic linkage.”

In some embodiments, the non-nucleotidic linker is a metal, including, without limitation, gold particles. In some other embodiments, the non-nucleotidic linker is a soluble or insoluble biodegradable polymer bead.

In yet other embodiments, the non-nucleotidic linker is an organic moiety having functional groups that permit attachment to the oligonucleotide. Such attachment preferably is by any stable covalent linkage. As a non-limiting example, the linker may be attached to any suitable position on the nucleoside. In some preferred embodiments, the linker is attached to the 3′-hydroxyl. In such embodiments, the linker preferably comprises a hydroxyl functional group, which preferably is attached to the 3′-hydroxyl by means of a phosphodiester, phosphorothioate, phosphorodithioate or non-phosphate-based linkages.

In some embodiments, the non-nucleotidic linker is a biomolecule, including, without limitation, polypeptides, antibodies, lipids, antigens, allergens, and oligosaccharides. In some other embodiments, the non-nucleotidic linker is a small molecule. For purposes of the invention, a small molecule is an organic moiety having a molecular weight of less than 1,000 Da. In some embodiments, the small molecule has a molecular weight of less than 750 Da.

In some embodiments, the small molecule is an aliphatic or aromatic hydrocarbon, either of which optionally can include, either in the linear chain connecting the oligonucleotides or appended to it, one or more functional groups selected from the group consisting of hydroxy, amino, thiol, thioether, ether, amide, thioamide, ester, urea, and thiourea. The small molecule can be cyclic or acyclic. Examples of small molecule linkers include, but are not limited to, amino acids, carbohydrates, cyclodextrins, adamantane, cholesterol, haptens and antibiotics. However, for purposes of describing the non-nucleotidic linker, the term “small molecule” is not intended to include a nucleoside.

In some embodiments, the small molecule linker is glycerol or a glycerol homolog of the formula HO—(CH₂)ₒ—CH(OH)—(CH₂)ₚ—OH, wherein o and p independently are integers from 1 to about 6, from 1 to about 4, or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6, or from 2 to about 4.

Some non-nucleotidic linkers according to the invention permit attachment of more than two oligonucleotides. For example, the small molecule linker glycerol has three hydroxyl groups to which oligonucleotides may be covalently attached. Some immunomers according to the invention, therefore, comprise more than two oligonucleotides linked to a non-nucleotidic linker. Some such immunomers comprise at least two IMOs, each having an accessible 5′ end.

In certain preferred embodiments, “siRNA” molecules useful in the methods according to the invention have one of the formulas set forth in U.S. Pat. No. 6,617,438, which is hereby incorporated by reference. Other siRNA molecules useful in the methods according to the invention include those with tolerated structural or chemical modifications. “Tolerated” modifications means those modifications that either increase stability or activity of the siRNA, or do not decrease the activity of the siRNA by more than 50%, preferably not more than 25%, more preferably not more than 10% and most preferably not more than 5%. For example, Chiu and Rana, RNA 9: 1034-1048 (2003) teach the introduction at various positions in the siRNA of adenine and guanine deoxynucleotides, 2′-O-Me ribonucleotides, phosporothioate ribonucleotides, 2′-fluoro-uridine, 2′-fluoro-cytidine, N³-methyl-uridine, 5-bromo-uridine, 5-iodo-uridine and 2,6-diamino-purine modifications are tolerated modifications. Braasch et al., Biochemistry 42: 7967-7975 (2003) teaches that locked nucleic acid (LNA) nucleotides are tolerated in siRNA. Harborth et al., Antisense and Nucleic Acid Drug Development 13: 83-105 teaches that 21-29 base pair hairpin siRNA was highly active and that 19-29 base pair hairpins are active when the 5′-end of the guide strand coincided with the 5′-end of the hairpin RNA. Holen et al., Nucleic Acids Research 31: 2401-2407 (2003) teaches that the antisense strand of siRNA alone is as active as double-stranded siRNA. Amarzguioui et al., Nucleic Acids Research 31: 589-595 (2003) teaches that G/C transversions and 2′-O-allylation are tolerated near the 5′ ends, but not the 3′ ends of siRNA. Each of these references are hereby incorporated by reference.

In the methods according to this aspect of the invention, administration of antisense oligonucleotides, siRNA and IMO can be by any suitable route, including, without limitation, parenteral, oral, sublingual, transdermal, topical, intranasal, aerosol, intraocular, intratracheal, intrarectal, vaginal, by gene gun, dermal patch or in eye drop or mouthwash form. Administration of the antisense oligonucleotides, siRNA and IMO can be carried out using known procedures at dosages and for periods of time effective to reduce symptoms or surrogate markers of the disease. When administered systemically, the therapeutic composition is preferably administered at a sufficient dosage to attain a blood level of from about 0.0001 micromolar to about 10 micromolar. For localized administration, much lower concentrations than this may be effective, and much higher concentrations may be tolerated. Preferably, a total dosage ranges from about 0.001 mg per patient per day to about 200 mg per kg body weight per day. It may be desirable to administer simultaneously, or sequentially a therapeutically effective amount of one or more of the therapeutic compositions of the invention to an individual as a single treatment episode.

“In combination with” means either simultaneously or sequentially. In the latter case, either the antisense oligonucleotide and/or siRNA may be administered either before or after the IMO.

In a second aspect, the invention provides a compostion of matter comprising a therapeutically effective amount of a VEGF expression-inhibiting antisense oligonucleotide and/or siRNA and a therapeutically effective amount of an IMO. All definitions are as described above.

In a third aspect, the invention provides a pharmaceutical formulation comprising a therapeutically effective amount of a VEGF expression-inhibiting antisense oligonucleotide and/or siRNA, a therapeutically effective amount of an IMO and a pharmaceutically acceptable carrier or diluent. As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990. All other definitions are as described above.

The following examples are intended to further illustrate certain preferred embodiments of the invention and are not intended to limit the scope of the claims.

### EXAMPLE 1

### Treatment Protocol

Mice are sensitized i.p. with 20 μg OVA in 100 μl PBS plus 100 μl alum solution on day 0 and 14. Mice are then treated i.n. with AS, IMO or SU1498 in 40 μl PBS on day 26, 27 and day 40, 41. Mice are then challenged with i.n. 10 μg OVA mixed with AS 5′-UGGCTTGAAGATGTACTCA (underlined nucleosides are 2′-O-methylribonucleosides) (SED. ID. NO.: 1) IMO 5′-TCTGACRTTCT-X-TCTRCAGTCT (R=2′-deoxy-7-deazaguanosine; X=glycerol linker) (SEQ. ID. NO.:2) or SU1498 (small molecule VEGF inhibitor) in 40 μl PBS on day 28 and 42. Mice are then challenged with i.n. 10 μg OVA in 40 μl PBS on day 49, and sacrificed on day 50 (24 h after last OVA challenge) and analyzed.

### EXAMPLE 2

### Prophylactic Protocol

Mice are i.p. injected with 20 μg OVA plus AS or/and IMO at various doses in 200 μl PBS/alum mixture on day 0, 7 and 14. Mice are i.n. challenged with 10 μg OVA in 40 μl PBS on day 21 and 22, and sacrificed on day 23 and analyzed.

What is claimed is:

1. A method for treating asthma and/or allergies comprising administering to a mammal having allergies and/or asthma a therapeutically effective amount of a VEGF expression-inhibiting antisense oligonucleotide and/or siRNA in combination with a therapeutically effective amount of an IMO.

2. A composition of matter comprising a therapeutically effective amount of a VEGF expression-inhibiting antisense oligonucleotide and/or siRNA and a therapeutically effective amount of an IMO.

3. A pharmaceutical formulation comprising a therapeutically effective amount of a VEGF expression-inhibiting antisense oligonucleotide and/or siRNA, a therapeutically effective amount of an IMO and a pharmaceutically acceptable carrier or diluent.
